(19) United States
(12) Patent Application Publication
(10) Pub. No.: US 2012/0101120 A1
(43) Pub. Date: Apr. 26, 2012

(54) PURE ISOMERS OF TRITOQUALINE

(76) Inventors: Michalis Nicolaou, San Diego, CA (US); Emile Loria, La Jolla, CA (US); Gaetan Terrasse, Saint-Valier (FR); Yves Trehin, Blesle (FR)

(21) Appl. No.: 13/217,072

(22) Filed: Aug. 24, 2011

Related U.S. Application Data
(63) Continuation of application No. 11/784,992, filed on Apr. 9, 2007, now abandoned.
(60) Provisional application No. 60/790,490, filed on Apr. 7, 2006.
(60) Provisional application No. 60/816,754, filed on Jun. 26, 2006.

(51) Int. Cl. A61K 31/4741; A61P 27/14; A61P 17/04; A61P 37/02; A61P 11/06; A61P 1/00; A61P 29/00; C07D 491/056; A61P 17/00

(52) U.S. Cl. 514/291; 546/90

(57) ABSTRACT

The invention provides an isolated stereoisomer of tritoqualine having the structure of FIG. 2 and FIG. 3.

This application is based on provisional applications, U.S. Ser. Nos. 60/790,490, filed Apr. 7, 2006, and 60/816,754, filed Jun. 26, 2006, the contents of which are hereby incorporated by reference, in their entirety, into this application.

Throughout this application various publications are referenced. The disclosures of these publications in their entireties are hereby incorporated by reference into this application in order to more fully describe the state of the art to which this invention pertains.

### FIELD OF THE INVENTION

The invention relates to novel tritoqualine isomers and uses thereof.

### BACKGROUND OF THE INVENTION

7-Amino-4,5,6-triethoxy-3-(5,6,7,8-tetrahydro-4-methoxy-6-methyl-1,3-dioxolo[4,5-g]isoquinolin-5-yl)phthalide or tritoqualine is a drug, currently formulated in 100 mg tablets and sold in pharmacies in Europe for the treatment of allergy.

The proposed mechanism of action of tritoqualine relates to the inhibition of histamine biosynthesis. More specifically, tritoqualine is an inhibitor of the enzyme histidine decarboxylase (HDC), which catalyzes histidine decarboxylation in vivo to produce histamine, an endogenous biogenic amine, plus carbon dioxide. Inhibiting histamine production in the body is proposed to ameliorate symptoms of allergy and other diseases that result from high histamine production.

It is well known that enzymes can be sensitive to the stereochemistry and chirality of inhibitory molecules. It is often the ease that one enantiomer of a compound will be a potent inhibitor of a target enzyme while the opposite enantiomer will be weak or inactive as an inhibitor.

Tritoqualine is not a pure product but is available as a mixture of isomers. The existing product and literature information does not disclose how many and which tritoqualine isomeric structures are present in the current product, and which isomers are active and are therapeutically useful inhibitors of the enzyme HDC. Isolating novel isomers of tritoqualine and identifying the most potent tritoqualine inhibitor would result in dose reduction and improved therapeutic profile compared to the currently marketed product.

### SUMMARY OF THE INVENTION

The invention provides a single diastereomeric structure comprised of two enantiomers, the RR and the SS. Embodiments of the two enantiomers of the invention include an isolated stereoisomer of tritoqualine having the structure D1 of FIG. 2 and an isolated stereoisomer of tritoqualine having the structure D2 of FIG. 3 and pharmaceutical compositions thereof. Preferred embodiments include pharmaceutical compositions, wherein the stereoisomer is essentially pure and free of other stereoisomers.

The invention also provides methods for treating diseases or disorders resulting from increased histamine levels comprising administering an effective amount of isomer D1 of FIG. 2 or isomer D2 of FIG. 3 to a subject.

The invention further provides a method of reducing histamine levels by inhibiting histidine decarboxylase comprising administering an effective amount of the isomer D1 of FIG. 2 or isomer D2 of FIG. 3 to a subject.

Also encompassed in this invention are methods for treating immune system diseases or disorders or other diseases that are directly or indirectly related to high histamine production, comprising administering to the subject an effective amount of isomers D1 or D2 of FIGS. 2 and 3, respectively.

### BRIEF DESCRIPTION OF THE FIGURES

FIG. 1 illustrates the chemical formula of tritoqualine (7-Amino-4,5,6-triethoxy-3-(5,6,7,8-tetrahydro-4-methoxy-6-methyl-1,3-dioxolo[4,5-g]isoquinolin-5-yl)phthalide)

FIG. 2 illustrates the sterical structure of the tritoqualine diastereomer D1.

FIG. 3 illustrates the sterical structure of the tritoqualine diastereomer D2.

FIG. 4 shows a chromatogram of the separation of tritoqualine stereoisomers via a chiral column. In the bottom part, the UV absorbance at 190 nm has been detected, while the top part depicts polarimetric detection at an averaged absorption in the range of 200-800 nm.

FIG. 5 shows a UV spectrum of each of the peaks of FIG. 4.

FIG. 6 illustrates the 3D-structures of the two stereoisomers (enantiomers) of FIGS. 4 and 5 as determined by X-Ray crystallography.

### DETAILED DESCRIPTION OF THE INVENTION

Definitions

As used herein, the term “stereoisomer” refers to isomeric molecules whose atomic connectivity is the same but whose atomic arrangement in space is different.

As used herein, the term “chiral” refers to a feature of an object (e.g. a molecule) which is nonsuperimposable on its mirror image. A molecule is chiral when it cannot be superimposed on its mirror image.

As used herein, the term “enantiomers” refers to two chiral stereoisomers that are related to each other by a reflection. They are mirror images of each other and their atoms are nonsuperposable. Enantiomers have, when present in a symmetric environment, identical chemical and physical properties except for their ability to rotate plane-polarized light by equal amounts but in opposite directions. A solution of equal parts of an optically-active isomer and its enantiomer is known as a “racemic solution” or “racemate” and has a net rotation of plane-polarized light of zero.

As used herein, the term “diastereomers” refers to stereoisomers which are not related through a reflection operation and are not mirror images of each other, for example, non-enantiomeric stereoisomers. Diastereomers seldom have the same physical properties.

As used herein, an “effective amount” of an isomer is defined as an amount that reduces histamine levels. Effective amount of a therapeutic agent (for example, D1 or D2) is dependant upon many factors including, but not limited to, the type of tissue affected, the type of disease being treated, the severity of the disease, a subject's health and response to the treatment with the agents. Accordingly, dosages of the agents can vary depending on each subject and the mode of administration.

As used herein, “purify” and “isolate” are used interchangeably. To purify or isolate means to remove contaminants from a compound of interest or to obtain or extract a substantially pure form of a compound of interest. For example, a stereoisomer may be isolated from a racemic mixture. In one embodiment, the isolated stereoisomer of tritoqualine has an RR configuration. In another embodiment, the isolated stereoisomer of tritoqualine has an SS configuration.

As used herein, “DMARDs” refer to a Disease Modifying Anti-Rheumatic Drug and can include, but are not limited to, dihydrofolic acid reductase inhibitors e.g., methotrexate; cyclophosphamide; cyclosporine; cyclosporin A; chloroquine; hydroxychloroquine; leflunomide; azathioprine; anakinra; and TNF blockers e.g., infliximab (REMICADE^{R}) or etanercept.

As used herein. “NSAIDs” refer to a Non-Steroidal Anti-Inflammatory Drug and reduce inflammatory reactions in a subject. NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, meloxicam and tramadol.

A “biodegradable carrier” comprises a composition that can be broken down and absorbed in an animal, such as a human.

As used herein, a “disease” refers to any deficiency, defect, pathology or abnormality in any bodily organs, tissues, cells, functions, bodily parts or activity in a subject, such as a human, and includes any disease, disorder, syndrome, and condition.

As used herein, “Treat,” “Treating” or “Treatment,” as used herein, covers any administration or application of remedies for disease in a mammal, including a human, and includes inhibiting the disease, arresting its development, preventing its progression, or relieving the symptoms, or ameliorating the effects of the disease for example, by causing regression, or restoring or repairing a lost, missing, or defective function; or stimulating an inefficient or absent process.

As used herein, a “pharmaceutically acceptable carrier” refers to a non-toxic solid, semisolid or liquid filler, diluent, encapsulating material or formulation auxiliary of any conventional type. A “pharmaceutically acceptable carrier” is non-toxic to recipients at the dosages and concentrations employed, and compatible with other ingredients of the formulation.

As used herein, the terms “subject,” “host,” “individual,” “animal,” and “patient,” used interchangeably herein, refer to mammals, including humans, and also include, but are not limited to, murines, simians, felines, canines, equines, bovines, porcines, ovines, caprins, rabbits, mammalian farm animals, mammalian sport animals, and mammalian pets. In many embodiments, the subjects will be humans. Animal models are of interest for experimental investigations, providing a model for treatment of human disease.

Compositions of the Invention

The invention provides purified stereoisomers of tritoqualine.

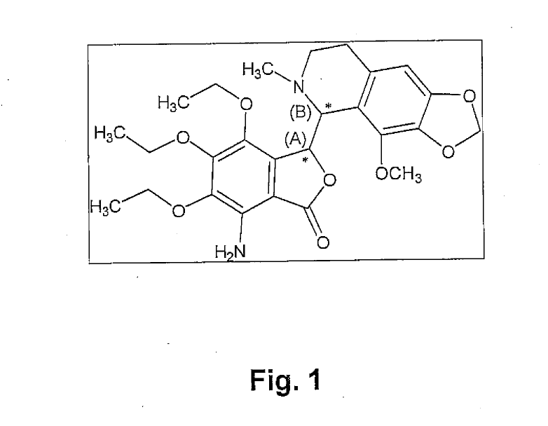

The known chemical structure of tritoqualine, illustrated in FIG. 1, is characterized by, amongst other structural features, the presence of two asymmetric carbons, A and B (marked with asterisk). Thus, depending on the method of synthesis, tritoqualine active pharmaceutical ingredient can be produced as either one or two diastereomeric structures each one comprising of its corresponding two mirror images, enantiomers. Thus, tritoqualine can exist as either two or four possible isomeric structures. Using the convention of R and S designation in each asymmetric carbon, one of the two possible diastereomeric structures will be comprised of the RR and SS enantiomers, and the other of RS and SR enantiomers.

In one embodiment, the isomers D1 or D2 are in salt form. In another embodiment, the isomers D1 or D2 are in hydrated form. In a further embodiment, the isomers D1 or D2 are administered with a pharmaceutically acceptable carrier.

Methods of the Invention

The present invention provides methods for treating diseases and disorders resulting from increased or elevated histamine levels. The method comprises administering an effective amount of isomers D1 or D2. Diseases and disorders with elevated histamine levels include but are not limited to allergic rhinitis, dermatitis, atopic dermatitis, urticaria, pruritus, eczema, allergic erythema and non allergic erythema, food allergy, asthma, inflammatory bowel disease such Irritable bowel disease, Crohn's disease, celiac disease, gastristis, GERD, oesophagitis and dyspepsia, Parkinson's diseases, myeloproliferative diseases,

The present invention further provides methods for treating immune system diseases and disorders comprising to the subject, an effective amount of isomers D1 or D2.

The present invention also provides a method of reducing histamine levels by inhibiting histidine decarboxylase. The method comprises administering an effective amount of the isomer D1 or D2 to a subject, thereby reducing histamine levels.

In one embodiment, the present invention provides methods for treating dermatitis including but not limited to chemical, cosmetic, acne aestivalis, anummular dermatitis, cercarial dermatitis, Duhring's Disease, atopic dermatitis, seborrhoeic dermatitis, Eczema and/or dyshidrosis, the method comprising administering to the subject, an effective amount of isomer D1 or D2.

In another embodiment, the present invention provides methods for treating conjunctivitis, allergic rhinitis, asthma, and/or allergy, the method comprising administering to the subject, an effective amount of isomer DI or D2.

The invention further provides pharmaceutical compositions that inhibit the enzyme Histidine decarboxylase (HDC). In one embodiment, the pharmaceutical composition that inhibits Histidine decarboxylase is the isomer D1. In another embodiment, the pharmaceutical composition that inhibits Histidine Carboxylase is the isomer D2. In a further embodiment, D1 or D2 are administered with a pharmaceutically acceptable carrier.

The pharmaceutically acceptable carriers include suitable carriers and adjuvants which include any material which when combined with the molecules of the invention (e.g. isomers D1 or D2) retain the molecule's activity, and is non-reactive with the subject's immune system. These carriers and adjuvants include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, phosphate buffered saline solution, water, emulsions (e.g. oil/water emulsion), salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances and polyethylene glycol. Other carriers may also include sterile solutions; tablets, including coated tablets and capsules. Typically such carriers contain excipients such as starch, milk, sugar (e.g. sucrose, glucose, maltose), certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods. Such compositions may also be formulated within various lipid compositions, such as, for example, liposomes as well as in various polymeric compositions, such as polymer microspheres.

Isomers of the invention may be administered by oral, intravenous, intramuscular, intraperitoneal, inhalation, nasal and subcutaneous methods, as well as implantable pump, continuous infusion, gene therapy, liposomes, suppositories, topical contact, vesicles, tablets, capsules, biodegradable polymers, hydrogels, controlled release patch and transdermal patch and injection methods.

In an embodiment of the invention, isomers D1 or D2 of the invention may be administered alone. In another embodiment, isomers D1 or D2 of the invention may be administered in conjunction with a second agent. Second agents can include the following: steroids, glucocorticoids, drug toxins, alkylating agents, anti-neoplastic drugs, enzymes, antibodies, conjugates, immunosuppressive agents, corticosteroids, DMARDs, nonsteroidal antiinflammatory drugs (NSAIDs), prednisone, azathioprine, methotrexate, TNFα blockers or antagonists, infliximab, any biological agent targeting an inflammatory cytokine, chloroquine, hydroxychloroquine, sulfasalazine (sulphasalazopryine), gold salts, etanercept, anakinra, cyclophosphamide, leflunomide, collagen, dnaJ, a molecule that blocks TNF receptors (e.g., pegsunercept), a molecule that blocks cytokine function (e.g., AMG719), a molecule that blocks LFA-1 function (e.g., efalizumab), acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, meloxicam, codeine phosphate, propoxyphene napsylate, oxycodone hydrochloride, oxycodone bitartrate, tramadol, dihydrofolic acid reductase inhibitor, cyclosporine, cyclosporin A or D-penicillamine. These isomers of the invention and the second agent may be administered sequentially or concomitantly. In GERD isomers of the invention may be administered with antisecretory product: ‘aluminum hydroxide, magnesium hydroxide, magnesium trisilicate, calcium carbonate and sodium bicarbonate and also with anti H2 product for example: cimetidine (Tagamet), ranitidine (Zantac), famotidine (Pepcid) et nizatidine (Axid) but also with Proton pomp inhibitor: Omeprazole (Prilosec), lansoprazole (Prevacid, Lansor), pantoprazole (Protonix), rebeprazole (Aciphex), and esoméprazole (Nexium) and also cisapride and also the CCK2 antagonists, and for asthma tritoqualine isomer may be administered with antileukotriènes for example: Montélukast, Pranlukast, Zafirlukast.

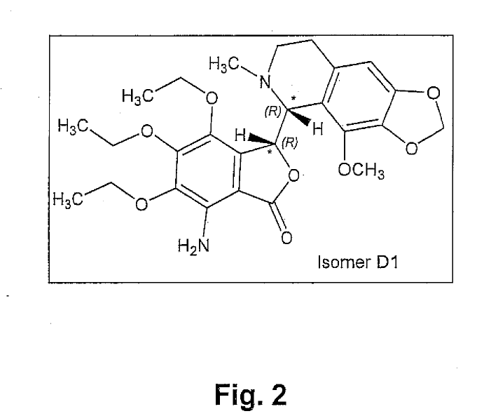

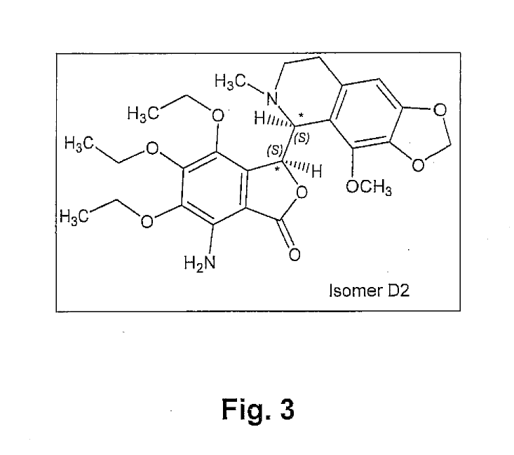

In one embodiment of the invention, an effective amount of isolated stereoisomer of tritoqualine having the structure D1 (FIG. 2) or D2 (FIG. 3) that may administered to a subject in order to treat diseases or disorders resulting from elevated histamine levels or to reduce histamine levels or to treat an immune system disease or disorder is about 0.1 to 300 mg/day, 0.1 to 150 mg/day, 0.1 to 100 mg/day, about 0.5 to 5 mg/day, about 5 to 300 mg/day, about 5 to 250 mg/day, about 5 to 200 mg/day, about 5 to 100 mg/day, about 10 to 100 mg/day, about 15 to 100 mg/day, about 20 to 100 mg/day, about 25 to 100 mg/day, about 30 to 100 mg/day, about 35 to 100 mg/day, about 40 to 100 mg/day, about 45 to 100 mg/day, about 50 to 100 mg/day, about 55 to 100 mg/day, about 60 to 100 mg/day, about 65 to 100 mg/day, about 70 to 100 mg/day, about 75 to 100 mg/day, about 80 to 100 mg/day, about 85 to 100 mg/day, about 90 to 100 mg/day, about 95 to 100 mg/day, about 5 to 150 mg/day, about 5 to 100 mg/day, about 5 to 50 mg/day, about 10 to 300 mg/day, about 10 to 250 mg/day, about 10 to 200 mg/day, about 10 to 150 mg/day, about 10 to 100 rag/day, about 10 to 50 mg/day, about 15 to 300 mg/day, about 15 to 250 mg/day, about 15 to 200 mg/day, about 15 to 150 mg/day, about 15 to 100 mg/day, about 15 to 50 mg/day, about 20 to 300 mg/day, about 20 to 250 mg/day, about 20 to 200 mg/day, about 20 to 150 mg/day, about 20 to 100 mg/day, about 20 to 50 mg/day, about 25 to 300 mg/day, about 25 to 250 mg/day, about 25 to 200 mg/day, about 25 to 150 mg/day, about 25 to 100 mg/day, about 25 to 50 mg/day, about 50 to 300 mg/day, about 50 to 250 mg/day, about 50 to 200 mg/day, about 50 to 150 mg/day, about 50 to 100 mg/day, about 5 to 10 mg/day, about 10 to 15 mg/day, about 15 to 20 mg/day, about 20 to 25 mg/day, about 25 to 30 mg/day, about 30 to 35 mg/day, about 35 to 40 mg/day, about 40 to 45 mg/day, about 45 to 50 mg/day, about 50 to 55 mg/day, about 55 to 60 mg/day, about 60 to 65 mg/day, about 65 to 70 mg/day, about 70 to 75 mg/day, about 75 to 80 mg/day, about 80 to 85 mg/day, about 85 to 90 mg/day, about 90 to 95 mg/day, about 95 to 100 mg/day, about 100 to 105 mg/day, about 105 to 110 mg/day, about 110 to 115 mg/day, about 115 to 120 mg/day, about 120 to 125 mg/day, about 125 to 130 mg/day, about 130 to 135 mg/day, about 135 to 140 mg/day, about 140 to 145 mg/day, about 145 to 150 mg/day, about 150 to 155 mg/day, about 155 to 160 mg/day, about 160 to 165 mg/day, about 165 to 170 mg/day, about 175 to 180 mg/day, about 180 to 185 mg/day, about 185 to 190 mg/day, about 190 to 195 mg/day, about 195 to 200 mg/day, about 200 to 205 mg/day, about 205 to 210 mg/day, about 210 to 215 mg/day, about 215 to 220 mg/day, about 220 to 225 mg/day, about 225 to 230 mg/day, about 230 to 235 mg/day, about 235 to 240 mg/day, about 240 to 245 mg/day, about 245 to 250 mg/day, about 250 to 255 mg/day, about 255 to 260 mg/day, about 260 to 265 mg/day, about 265 to 270 mg/day, about 270 to 275 mg/day, about 275 to 280 mg/day, about 280 to 285 mg/day, about 285 to 290 mg/day, about 290 to 295 mg/day, or about 295 to 300 mg/day. It would be clear to one skilled in the art that dosage range will vary depending on the intensity and duration of the diseases. Further, it would be clear to one skilled in the art that dosage range will vary depending on the age, sex, height and/or weight of the subject and the stage at which the disease is diagnosed.

The following example is presented to illustrate the present invention and to assist one of ordinary skill in making and using the same. The examples are not intended in any way to otherwise limit the scope of the invention.

### EXAMPLE 1

Materials and Methods

Extraction of tritoqualine from tablets: Forty 100 mg tritoqualine tablets were crushed using mortar and pestle and the white powder was transferred to an Erlenmeyer flask. Addition of 400 mL ethyl acetate resulted in the formation of a fine white suspension. The suspension was allowed to stir for 1 hour under ambient conditions. Filtration of all insoluble matter, removal of solvent by rotary evaporation afforded a white crystalline solid. This solid was then dissolved in approximately 100 mL of dichloromethane. Hexane was added to the above solution until it became cloudy. After overnight storage at room temperature, Tritoqualine crystalline material formed at the bottom of the glass affording 3.5 g of pure tritoqualine.

Analytical Separation and Isolation of Tritoqualine Stereoisomers:

Thin layer chromatography: various proportions of ethyl acetate/hexane, dichloromethane/hexane, and ethyl acetate dichloromethane were used in conjunction with silca-based thin layer chromatography to identify the number of compounds available in the mixture. In all cases of mobile phase mixtures, there was only one single spot observed (seen under UV light) indicating the presence of only one diastereomer. The two enantiomers comprising the diastereomer could not be resolved using silica-based thin layer chromatography.

HPLC separation of tritoqualine enantiomers: HPLC separation was conducted using an Agilent 1100 HPLC system equipped with a quaternary pump, injector, diode array detector and a Jasco OR-990 polarimetric detector. The successful chromatographic separation utilized the chiral HPLC column CHIRALPAK®IA (250 mm, 4.6 mm, 5 μm) with the following conditions: mobile phase: n-heptane/dichloromethane 60:40; flow rate 1 ml/min; temp 25° C.; tritoqualine concentration injected was 8 g/l in mobile phase; injection volume 1 μl; UV detection:290 nm. UV spectra for each enantiomer were obtained using the diode array detector and absorption of polarized light using a polarimetric detector.

HPLC purification of tritoqualine enantiomers: Purification of each tritoqualine enantiomer was conducted using a similar Agilent HPLC with a preparatory chiral column CHIRALPAK®IA (250 mm, 4.6 mm, 5 μm). Mobile phase: n-heptane dichloromethane 60:40; flow rate 20 mL/min; temp 25° C., UV detection 250 nm. Each enantiomer was collected as was eluted from the column. To ensure purity of each enantiomer HPLC analysis using the analytical column CHIRALPAK®IA (250 mm, 4.6 mm, 5 μm), mobile phase: n-heptane/dichloromethane 60:40; flow rate 1 ml/min; temp 25° C.; UV detection:250 nm. Enantiomer A eluted at retention time of −5.95 min and enantiomer B at retention time of 7.19 mins. Chemical purities for each isolated compound exceeded the 99.5%. Enantiomeric excess for enantiomer A was 99.5% and enantiomer B was 99.0%. Solvent removal afforded each isolated isomer as an amorphous white powder.

Characterization of the Commercial Mixture of Tritoqualine and of each Isolated Enantiomer by NMR.

¹H NMR spectra were recorded on a Brucker AMX 500 (500 MHz). Chemical shifts are expressed in parts per million (δ) relative to residual solvents as internal standards.

¹H NMR characterization of the commercial tritoqualine product isolated from tablets: ¹H NMR (CDCl₃) δ 6.36 (1H, s), 5.88 (2H, m), 5.59 (1H, d, J=1.71 Hz), 5.03 (2H, s), 4.54 (1H, s), 4.08 (9H, m), [3.08 (1H, m), 2.76 (1H, m), 2.56 (1H, m), 2.43 (1H, m)], 2.14 (3H, s), 1.39-1.45 (9H, m).

¹H NMR characterization of isolated enantiomer A: ¹H NMR (CDCl₃) δ Ar 6.35 (1H, s,), O—, 5.87 (2H, m), 5.58 (1H, s), 5.02 (2H, s), 4.54 (1H, s), 4.08 (9H, m), [3.04 (1H, m), 2.79 (1H, m), 2.55 (1H, m), 2.41 (1H, m)], 2.13 (3H, s), 1.37-1.45 (9H, m).

¹H NMR characterization of isolated enantiomer B: ¹H NMR (CDCl₃) δ 6.36 (1H, s,), 5.88 (2H, m), 5.58 (1H, d, J=1.71 Hz), 5.02 (2H, s), 4.54 (1H, s), OCH₃ 4.07 (9H, m), 3.04 (1H, m), 2.77 (1H, m), 2.55 (1H, m), 2.41 (1H, m), 2.13 (3H, s), 1.37-1.45 (9H, m).

Crystallography

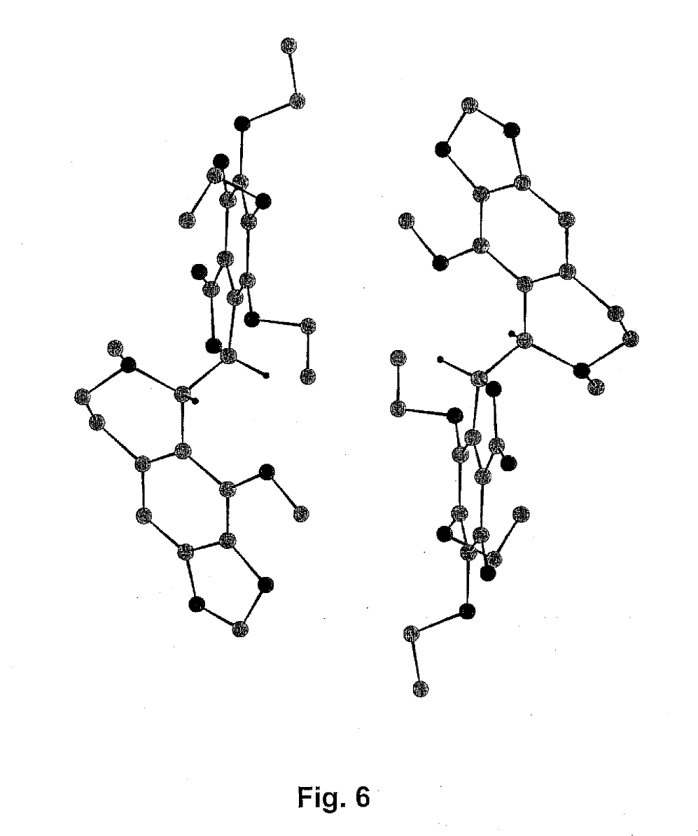

A crystal of tritoqualine, afforded by the recrystallization procedure described above, was chosen for X-Ray crystallography. The crystal structure of commercial tritoqualine was determined by an expert crystallographer. The data is reported in Tables S1-S5 and a picture of the existing structures is illustrated in FIG. 6 below.

Crystal Structure Determination C₂₆H₃₂N₂O₈

The Bruker X8-APEX X-ray diffraction instrument with Mo-radiation was used for data collection. All data frames were collected at low temperatures (T=90 K), Using an ω, φ-scan mode (0.3° ω-scan width, hemisphere of reflections), and integrated using a Bruker SAINTPLUS software package. The intensity data were corrected for Lorentzian polarization. Absorption corrections were performed using the SADABS program. The SIR97 was used for direct methods of phase determination, and Bruker SHELXTL software for structure refinement and difference Fourier maps. Atomic coordinates, isotropic and anisotropic displacement parameters, of all the non-hydrogen atoms were refined, by means of a full matrix least-squares procedure on F². All H-atoms were included in the refinement, in calculated positions riding on the C atoms, with W[iso] fixed at 20% higher, than isotropic parameters of carbons atoms which they were attached. Drawing of molecule was performed using Ortep 3.

Crystal and structure parameters: size 0.38×0.20×0.10 mm³, monoclinic, space group P2(1)/n, a=16.7348(6) Å, b=7.8819(3) Å, c=18.5117(6) Å, α=90.0° β=985090(10)° γ=90.0°, V=2414.85(15) Å³, ρ_{calcd}=1.377 g/cm³, 2θₘₐₓ=65.26°, Mo-radiation (λ=0.71073 Å), low temperature=90(2) K, reflections collected=33322, independent reflections=8434 (Rᵢₙₜ=0.0372, R_{sig}=0.0382), 6524 (77.4%) reflections were greater than 2σ(I), index ranges 25<=h<=24, −11<=k<=10, −27<=l<=25, absorption coefficient μ=0.102 mm⁻¹: max/min transmission=0.9898 and 0.9621, 399 parameters were refined and converged at R1=0.0493, wR2=0.1210, with intensity I>2σ(I), the final difference map was 0.431 and −0.272 e.Å⁻³.

Mass Spectrometry

Mass spectrometry results showed molecular ion peaks for each enantiomer to be 500. The mass spectrometry data was recorded on Applied Biosystems PI 100 electrospray mass spectrometer. The samples were run in positive mode and (M⁺+1) values are reported 501.6 for enantiomer A and 501.5 for enantiomer B.

Results and Discussion

Separation, Characterization and Isolation of Tritoqualine Enantiomers:

Silica-based thin layer chromatography, was not able to separate and resolve any tritoqualine diastereomers. In general diastereomeric compounds can be separated in silica-based thin layer chromatography. Enantiomers, on the other hand cannot be separated by silica-based chromatography. A chiral solid phase is necessary to separate and resolve enantiomers. Therefore, it was postulated that the commercial tritoqualine material was, probably, a mixture of enantiomers.

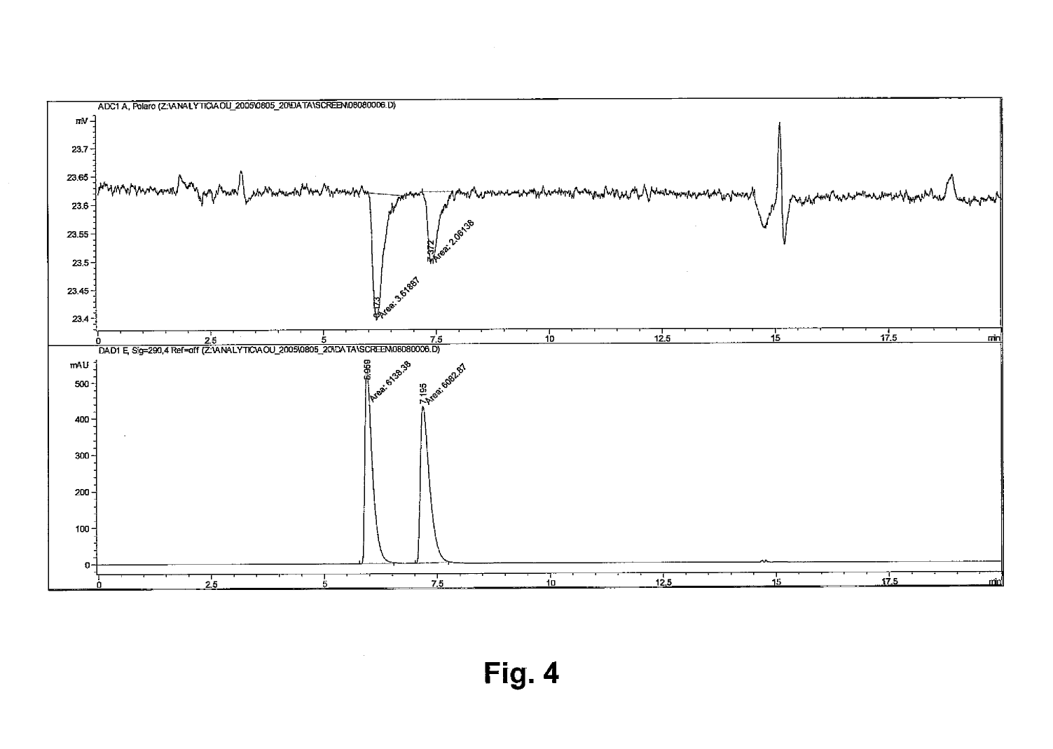

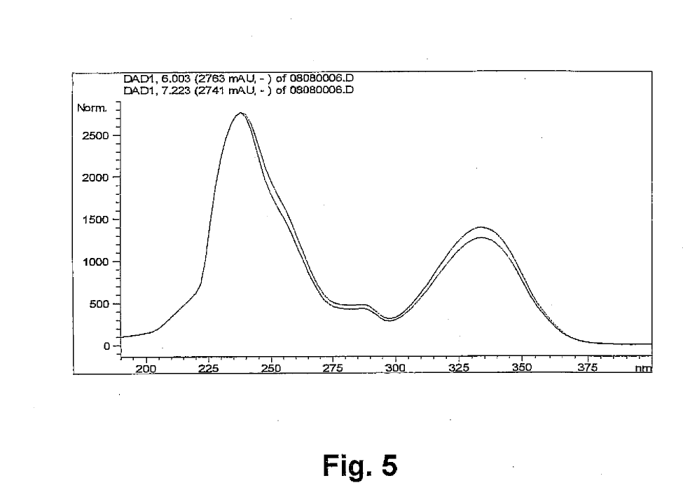

Chiral chromatography was employed in order to test commercial tritoqualine (two chiral centers) for the presence of enantiomers. FIG. 4 (bottom part) illustrates a representative chromatogram of tritoqualine chromatographed on a chiral column. Clearly, two distinct and well resolved peaks of approximately the same area could be identified, at 5.95 and 7.19 minutes respectively. Polarimetric detection (FIG. 4, top part) indicates that each peak on the chromatogram absorbs polarized light suggesting that each molecule eluting from the chiral column is an optically active compound. However, the polarimetric detector, in contrast to the standard polarimeters, does not measure the sign of the rotatory power at a given wavelength, but only gives an average response over a range of wavelengths (200-800 nm). As the sign of the rotatory power may change depending on the wavelength for the same isomer (for certain compounds), especially for compounds having UV absorption at high wavelengths (>300 nm) which is the case of tritoqualine (FIG. 5), it was not possible to draw conclusions by this technique beyond the notion that each peak represents an optical isomer. From the diode array detector available on the HPLC setup the UV spectrum of each peak was obtained as shown on FIG. 5. Both compounds show almost identical UV spectra, which is the case of enantiomers. To further confirm the presence of enantiomers, ¹HNMR spectra of the mixture and of the individual components are identical. If two optically active diastereomers were present in the mixture, then two sets of peaks for each diastereomer would have been expected

Diastereomer Identification in Commercial Tritoqualine:

The tritoqualine structure contains two chiral centers (FIG. 1). Thus, there could only be two possible diastereomeric structures. One comprised of the enantiomers RR and SS and a-second comprised of the enantiomers RS and SR.

Based on the data generated above, the only reasonable conclusion was that commercial tritoqualine is a single diastereomeric structure. The challenge to find whether commercial tritoqualine is the RR/SS or the RS/SR remains.

To solve this issue, a single crystal from the recrystallized tritoqualine was identified and the crystal structure was determined by an expert crystallographer. The crystallography data, indicates that on the single tritoqualine crystal there are two molecules present that are enantiomers of a single diastereomer. The two enantiomers bear the RR and the SS configuration.

All relevant information is shown on Tables S1-S5 and the molecular structures of the two enantiomers are illustrated on FIG. 6.

Isolation of Tritoqualine Enantiomers for the Purposes of Biological Activity Determination:

Using the preparatory chiral column CHIRALPAK®IA (250 mm, 4.6 mm, 5 μm) and the HPLC system described above, the two enantiomers, enantiomer A and B have been successfully isolated as amorphous white powders.

Purification of Human Histidine Decarboxylase

The DNA encoding for residues 1-512 of human HDC was subcloned in the pGEX-6P-1 vector (GE-Healthcare). The recombinant plasmid transformed into the Escherichia coli BL21(DE3)pLysS strain. Transformed cultures were induced to express the HDC 1/512, which was purified by affinity chromatography using Glutathione sepharose (GE-Healthcare). 1/512 HDC was released from the fusion protein bound to the affinity chromatography support by digestion with the Pre-Scission™ protease (GE-Healthcare). The final preparations were dissolved in 50 mM potassium phosphate, 0.1 mM PLP, pH 7.0. Purity of the HDC 1/512 construct was checked by Coomassie blue staining and Western blotting, and was higher than 95% in the final preparations.

Human-HDC Activity Determination

HDC activity was assayed, as described in Engel at al. (1996) Biochem J. 320: 365-368, by measuring the production of ¹⁴CO2 from L-[U-¹⁴C]histidine (GE-Healthcare) in a mixture containing 0.2 mM dithiothreitol, 10 μM PLP, 10 mg/ml poly(ethylene glycol)-300, 100 mM potassium phosphate, pH 6.8, and purified protein in a total volume of 100 μL. When recombinant HDC was used, the concentration of L-[U-¹⁴C]histidine was 13.3 μM (with ⅓ isotopic dilution). The released ¹⁴CO2 was measured as previously described for HDC activity determinations (Urdiales et al. (1992) FEBS Lett. 305, 260-264).

Assessment of Inhibitory Activity of each Isomeric Component, Versus the Mixture:

10 μM concentration of each isomer, A and B (A corresponds to the isolated pure isomer eluting at 5.9 minutes, B corresponds to the isolated pure isomer eluting at 7.1 minutes of the chromatogram shown in FIG. 4 (bottom)) and their corresponding racemic mixture (starting material prior to separating the individual isomers, indicated as A+B) along with 4 μg of recombinant human HDC were used to assess the inhibitory effect of each isomer and the mixture on the enzymatic conversion of histidine to histamine. Table 1 summarizes results obtained. Results are presented as means of duplicates samples. As shown in Table 1, the pure isomers (isomer A and isomer B) have more activity compared to the racemic mix (A+B).

TABLE 1
Effect of compound A, B and A + B on activity of recombinant
HDC at micromolar concentration.

| | | | Specific activ- | | |
|---|---|---|---|---|---|
| | | Activity | ity (μmole/ | % of | % of |
| Sample | DPM | (μmole/h) | h · mg prot) | control | inhibition |
| Control | 12340 | 0.35 | 87.00 | 100.00 | |
| Isomer A (10 | 8895 | 0.25 | 62.71 | 72.08 | 27.92 |
| μM final) | | | | | |
| Isomer B (10 | 7831 | 0.22 | 55.21 | 63.46 | 36.54 |
| μM final) | | | | | |
| Racemic mix | 10176 | 0.29 | 71.74 | 82.46 | 17.54 |
| A + B (10 μM | | | | | |
| final) | | | | | |

TABLE S1
Crystal data and structure refinement.

| | | |
|---|---|---|
| Empirical formula | C₂₆H₃₂N₂O₈ | |
| Formula weight | 500.54 | |
| Temperature | 90(2) K | |
| Wavelength | 0.71073 Å | |
| Crystal system | Monoclinic | |
| Space group | P2(1)/n | |
| Unit cell dimensions | a = 16.7348(6) Å | □ = 90° |
| | b = 7.8819(3) Å | □ = 98.5090(10)° |
| | c = 18.5117(6) Å | □ = 90° |
| Volume | 2414.85(15) Å³ | |
| Z | 4 | |
| Density (calculated) | 1.377 Mg/m³ | |
| Absorption coefficient | 0.102 mm⁻¹ | |
| F(000) | 1064 | |
| Crystal size | 0.38 × 0.20 × 0.10 mm³ | |
| Theta range for data | 2.22 to 32.63° | |
| collection | | |
| Index ranges | −25 <= h < = 24, −11 <= k <= 10, | |
| | −27 <= 1 <= 25 | |
| Reflections collected | 33322 | |
| Independent reflections | 8434 [R(int) = 0.0372] | |
| Completeness to theta = | 95.7% | |
| 32.63° | | |
| Absorption correction | Sadabs | |
| Max. and min. trans- | 0.9898 and 0.9621 | |
| mission | | |
| Refinement method | Full-matrix least-squares on F² | |
| Data/restraints/para- | 8434/0/399 | |
| meters | | |
| Goodness-of-fit on F² | 1.021 | |
| Final R indices [I > | R1 = 0.0493, wR2 = 0.1210 | |
| 2sigma(I)] | | |
| R indices (all data) | R1 = 0.0677, wR2 = 0.1309 | |
| Largest diff. peak | 0.431 and −0.272 e.Å⁻³ | |
| and hole | | |

TABLE S2
Atomic coordinates (×10⁴) and equivalent isotropic
displacement parameters (Å² × 10³) U(eq) is
defined as one third of the trace of the orthogonalized U^{ij} tensor.

| | x | y | z | U(eq) |
|---|---|---|---|---|
| N(1) | 4187(1) | 505(1) | 7064(1) | 15(1) |
| N(2) | 2469(1) | 6526(1) | 7408(1) | 22(1) |
| O(1) | 4787(1) | 3926(1) | 7526(1) | 17(1) |
| O(2) | 8074(1) | 270(1) | 7914(1) | 30(1) |
| O(3) | 7817(1) | 1237(1) | 6723(1) | 25(1) |
| O(4) | 4067(1) | 5361(1) | 8264(1) | 22(1) |
| O(5) | 6084(1) | 1991(1) | 5982(1) | 19(1) |
| O(6) | 3737(1) | 2797(1) | 5250(1) | 20(1) |
| C(1) | 4114(1) | 4785(1) | 7666(1) | 16(1) |
| C(2) | 3536(1) | 4844(1) | 6994(1) | 16(1) |
| C(3) | 2767(1) | 5590(1) | 6881(1) | 17(1) |
| C(4) | 2330(1) | 5387(2) | 6178(1) | 20(1) |
| C(5) | 2649(1) | 4475(2) | 5638(1) | 20(1) |
| C(6) | 3433(1) | 3771(2) | 5762(1) | 18(1) |
| C(7) | 3863(1) | 3974(1) | 6453(1) | 15(1) |
| C(8) | 4689(1) | 3349(1) | 6771(1) | 15(1) |
| C(9) | 4825(1) | 1417(1) | 6743(1) | 14(1) |
| C(10) | 5687(1) | 1028(1) | 7087(1) | 15(1) |
| C(11) | 6312(1) | 1350(1) | 6668(1) | 16(1) |
| C(12) | 7096(1) | 1016(2) | 6998(1) | 19(1) |
| C(13A) | 8410(5) | 428(8) | 7244(5) | 32(1) |
| C(13B) | 8434(13) | 880(20) | 7355(13) | 43(4) |
| C(14) | 7249(1) | 432(2) | 7708(1) | 22(1) |
| C(15) | 6658(1) | 112(2) | 8126(1) | 23(1) |
| C(16) | 5857(1) | 420(2) | 7800(1) | 18(1) |
| C(17) | 5150(7) | 4(2) | 8183(1) | 22(1) |
| C(18) | 4479(1) | −737(2) | 7633(1) | 20(1) |
| C(19) | 6648(1) | 1882(2) | 5474(1) | 24(1) |
| C(20A) | 3999(3) | 3784(5) | 4676(2) | 27(1) |
| C(20B) | 3831(7) | 3420(13) | 4531(6) | 34(2) |
| C(21) | 4431(1) | 2557(2) | 4224(1) | 28(1) |
| O(7A) | 2204(2) | 4414(3) | 4960(2) | 18(1) |
| (C22A) | 1509(1) | 3252(2) | 4911(1) | 18(1) |
| C(23A) | 1771(2) | 1542(3) | 4685(1) | 44(1) |
| O(7B) | 2188(7) | 3888(8) | 4956(7) | 28(2) |
| C(22B) | 1726(6) | 2260(20) | 5010(5) | 93(5) |
| C(23B) | 1508(4) | 1574(8) | 4304(4) | 36(1) |
| O(8A) | 1549(3) | 6094(11) | 6050(4) | 20(1) |
| C(24A) | 1469(6) | 7557(10) | 5574(5) | 22(1) |
| O(8B) | 1575(10) | 5820(30) | 6002(10) | 28(4) |
| C(24B) | 1476(16) | 7230(30) | 5472(14) | 28(3) |
| C(25) | 578(1) | 7703(2) | 5283(1) | 29(1) |
| C(26) | 3628(1) | −354(2) | 6496(1) | 20(1) |

TABLE S3
Bond lengths [Å] and angles [°].

| | | | |
|---|---|---|---|
| N(1)—C(26) | 1.4661(14) | N(1)—C(18) | 1.4673(15) |
| N(1)—C(9) | 1.4828(14) | N(2)—C(3) | 1.3728(16) |
| N(2)—HN1 | 0.885(18) | N(2)—HN2 | 0.96(2) |
| O(1)—C(1) | 1.3714(13) | O(1)—C(8) | 1.4559(14) |
| O(2)—C(13B) | 1.36(2) | O(2)—C(14) | 1.3841(13) |
| O(2)—C(13A) | 1.442(10) | O(3)—C(12) | 1.3866(14) |
| O(3)—C(13A) | 1.424(9) | O(3)—C(13B) | 1.47(2) |
| O(4)—C(1) | 1.2090(15) | O(5)—C(11) | 1.3678(14) |
| O(5)—C(19) | 1.4294(14) | O(6)—C(6) | 1.3759(14) |
| O(6)—C(20A) | 1.436(5) | O(6)—C(20B) | 1.448(12) |
| C(1)—C(2) | 1.4590(15) | C(2)—C(7) | 1.3898(16) |
| C(2)—C(3) | 1.4035(14) | C(3)—C(4) | 1.4036(17) |
| C(4)—O(8B) | 1.305(17) | C(4)—C(5) | 1.4000(18) |
| C(4)—O(8A) | 1.409(6) | C(5)—O(7A) | 1.361(4) |
| C(5)—C(6) | 1.4115(15) | C(5)—O(7B) | 1.454(11) |
| C(6)—C(7) | 1.3801(16) | C(7)—C(8) | 1.5043(14) |
| C(8)—C(9) | 1.5419(15) | C(9)—C(10) | 1.5197(14) |
| C(10)—C(16) | 1.3930(16) | C(10)—C(11) | 1.4144(15) |
| C(11)—C(12) | 1.3880(14) | C(12)—C(14) | 1.3810(18) |
| C(14)—C(15) | 1.3662(19) | C(15)—C(16) | 1.4065(15) |
| C(15)—H(15) | 0.9500 | C(16)—C(17) | 1.5032(17) |
| C(17)—C(18) | 1.5168(17) | C(20A)—C(21) | 1.529(5) |
| C(20B)—C(21) | 1.402(13) | O(7A)—C(22A) | 1.473(4) |
| C(22A)—C(23A) | 1.496(3) | O(7B)—C(22B) | 1.509(17) |
| C(22B)—C(23B) | 1.412(10) | O(8A)—C(24A) | 1.445(12) |
| C(24A)—C(25) | 1.512(10) | O(8B)—C(24B) | 1.47(3) |
| C(24B)—C(25) | 1.54(3) | | |
| C(26)—N(1)—C(18) | 108.46(9) | C(26)—N(1)—C(9) | 110.92(9) |
| C(18)—N(1)—C(9) | 115.39(8) | C(3)—N(2)—HN1 | 115.2(12) |
| C(3)—N(2)—HN2 | 114.0(11) | HN1—N(2)—HN2 | 116.8(16) |
| C(1)—O(1)—C(8) | 110.92(8) | C(13B)—O(2)—C(14) | 107.1(9) |
| C(14)—O(2)—C(13A) | 104.7(3) | C(12)—O(3)—C(13A) | 104.7(4) |
| C(12)—O(3)—C(13B) | 103.4(9) | C(11)—O(5)—C(19) | 117.87(9) |
| C(6)—O(6)—C(20A) | 113.09(18) | C(6)—O(6)—C(20B) | 123.1(5) |
| O(4)—C(1)—O(1) | 121.60(10) | O(4)—C(1)—C(2) | 130.14(10) |
| O(1)—C(1)—C(2) | 108.26(10) | C(7)—C(2)—C(3) | 123.33(10) |
| C(7)—C(2)—C(1) | 108.53(9) | C(3)—C(2)—C(1) | 128.12(11) |
| N(2)—C(3)—C(2) | 122.73(11) | N(2)—C(3)—C(4) | 121.72(10) |
| C(2)—C(3)—C(4) | 115.48(11) | O(8B)—C(4)—C(5) | 114.4(10) |
| O(8B)—C(4)—C(3) | 123.7(9) | C(5)—C(4)—C(3) | 121.34(10) |
| C(5)—C(4)—O(8A) | 121.6(3) | C(3)—C(4)—O(8A) | 117.0(3) |
| O(7A)—C(5)—C(4) | 117.59(17) | O(7A)—C(5)—C(6) | 120.30(18) |
| C(4)—C(5)—C(6) | 121.85(11) | C(4)—C(5)—O(7B) | 125.1(5) |
| C(6)—C(5)—O(7B) | 112.2(4) | O(6)—C(6)—C(7) | 120.53(9) |
| O(6) C(6)—C(5) | 122.43(10) | C(7)—C(6)—C(5) | 116.79(11) |
| C(6)—C(7)—C(2) | 121.17(9) | C(6)—C(7)—C(8) | 130.40(10) |
| C(2)—C(7)—C(8) | 108.42(9) | O(1)—C(8)—C(7) | 103.86(8) |
| O(1)—C(8)—C(9) | 110.19(9) | C(7)—C(8)—C(9) | 116.21(9) |
| N(1)—C(9)—C(10) | 115.37(9) | N(1)—C(9)—C(8) | 110.29(8) |
| C(10)—C(9)—C(8) | 108.67(8) | C(16)—C(10)—C(11) | 121.12(9) |
| C(16)—C(10)—C(9) | 121.03(10) | C(11)—C(10)—C(9) | 117.83(9) |
| O(5)—C(11)—C(12) | 126.46(10) | O(5)—C(11)—C(10) | 116.61(9) |
| C(12)—C(11)—C(10) | 116.92(10) | C(14)—C(12)—O(3) | 110.05(9) |
| C(14)—C(12)—C(11) | 120.74(11) | O(3)—C(12)—C(11) | 129.17(11) |
| O(3)—C(13A)—O(2) | 107.6(5) | O(2)—C(13B)—O(3) | 109.6(14) |
| C(15)—C(14)—C(12) | 123.63(10) | C(15)—C(14)—O(2) | 127.12(12) |
| C(12)—C(14)—O(2) | 109.24(11) | C(14)—C(15)—C(16) | 116.61(11) |
| C(10)—C(16)—C(15) | 120.97(11) | C(10)—C(16)—C(17) | 117.20(9) |
| C(15)—C(16)—C(17) | 121.72(11) | C(16)—C(17)—C(18) | 108.89(10) |
| N(1)—C(18)—C(17) | 111.09(10) | O(6)—C(20A)—C(21) | 106.4(3) |
| C(21)—C(20B)—O(6) | 113.0(7) | C(5)—O(7A)—C(22A) | 113.4(3) |
| O(7A)—C(22A)—C(23A) | 108.44(19) | C(5)—O(7B)—C(22B) | 115.0(8) |
| C(23B)—C(22B)—O(7B) | 109.2(9) | C(4)—O(8A)—C(24A) | 114.7(6) |
| O(8A)—C(24A)—C(25) | 106.0(6) | C(4)—O(8B)—C(24B) | 112.0(16) |
| O(8B)—C(24B)—C(25) | 110.1(18) | | |

TABLE S4
Anisotropic displacement parameters (Å² × 10³).
The anisotropic displacement factor exponent takes the form:
−2π²[h²a*²U¹¹ + . + 2 h k a* b* U¹²]

| | U¹¹ | U²² | U³³ | U²³ | U¹³ | U¹² |
|---|---|---|---|---|---|---|
| N(1) | 15(1) | 14(1) | 16(1) | 1(1) | 4(1) | −2(1) |
| N(2) | 19(1) | 20(1) | 29(1) | 1(1) | 10(1) | 3(1) |
| O(1) | 16(1) | 16(1) | 19(1) | −2(1) | 1(1) | 1(1) |
| O(2) | 17(1) | 36(1) | 35(1) | −1(1) | −6(1) | 8(1) |
| O(3) | 12(1) | 27(1) | 36(1) | 3(1) | 2(1) | 2(1) |
| O(4) | 26(1) | 20(1) | 21(1) | −3(1) | 5(1) | 0(1) |
| O(5) | 15(1) | 23(1) | 19(1) | 5(1) | 5(1) | 3(1) |
| O(6) | 22(1) | 22(1) | 17(1) | 1(1) | 5(1) | 2(1) |
| C(1) | 17(1) | 13(1) | 21(1) | 0(1) | 4(1) | −1(1) |
| C(2) | 14(1) | 14(1) | 20(1) | 2(1) | 3(1) | 0(1) |
| C(3) | 15(1) | 15(1) | 23(1) | 4(1) | 7(1) | 1(1) |
| C(4) | 13(1) | 23(1) | 24(1) | 8(1) | 5(1) | 3(1) |
| C(5) | 14(1) | 27(1) | 19(1) | 6(1) | 2(1) | 1(1) |
| C(6) | 15(1) | 20(1) | 18(1) | 2(1) | 3(1) | 1(1) |
| C(7) | 13(1) | 14(1) | 18(1) | 3(1) | 3(1) | 1(1) |
| C(8) | 13(1) | 14(1) | 17(1) | 1(1) | 2(1) | 0(1) |
| C(9) | 13(1) | 14(1) | 15(1) | 1(1) | 2(1) | 0(1) |
| C(10) | 14(1) | 13(1) | 17(1) | −1(1) | 1(1) | 2(1) |
| C(11) | 15(1) | 14(1) | 19(1) | 0(1) | 1(1) | 2(1) |
| C(12) | 14(1) | 16(1) | 27(1) | 0(1) | 2(1) | 2(1) |
| C(13A) | 14(1) | 36(2) | 45(2) | 5(2) | −3(1) | 5(1) |
| C(13B) | 16(3) | 64(10) | 45(8) | 23(7) | −3(4) | −2(7) |
| C(14) | 17(1) | 19(1) | 28(1) | −3(1) | −5(1) | 5(1) |
| C(15) | 24(1) | 23(1) | 19(1) | 1(1) | −3(1) | 6(1) |
| C(16) | 21(1) | 17(1) | 16(1) | −1(1) | 1(1) | 4(1) |
| C(17) | 25(1) | 24(1) | 16(1) | 4(1) | 3(1) | 4(1) |
| C(18) | 24(1) | 17(1) | 19(1) | 3(1) | 7(1) | 0(1) |
| C(19) | 22(1) | 31(1) | 23(1) | 1(1) | 10(1) | 4(1) |
| C(20A) | 42(2) | 18(1) | 24(2) | −5(1) | 18(1) | −4(1) |
| C(20B) | 46(5) | 34(6) | 23(5) | 13(4) | 10(3) | 19(4) |
| C(21) | 34(1) | 25(1) | 28(1) | −3(1) | 14(1) | 0(1) |
| O(7A) | 14(1) | 21(1) | 18(1) | 3(1) | 0(1) | −2(1) |
| C(22A) | 11(1) | 17(1) | 24(1) | 4(1) | −1(1) | 2(1) |
| C(23A) | 35(1) | 28(1) | 67(2) | −2(1) | 3(1) | −2(1) |
| O(7B) | 25(2) | 32(4) | 23(2) | 6(4) | −5(2) | 2(3) |
| C(22B) | 55(5) | 191(16) | 36(5) | −50(8) | 13(4) | −42(8) |
| C(23B) | 30(3) | 25(3) | 54(4) | 5(3) | 9(3) | 4(2) |
| O(8A) | 10(1) | 24(2) | 26(1) | 7(1) | 4(1) | 8(1) |
| C(24A) | 19(1) | 22(2) | 25(2) | 9(1) | 8(1) | 7(2) |
| O(8B) | 26(3) | 28(7) | 36(5) | 24(5) | 22(3) | 18(3) |
| C(24B) | 16(3) | 35(9) | 36(8) | 13(5) | 10(5) | 14(5) |
| C(25) | 20(1) | 36(1) | 29(1) | 11(1) | 3(1) | 9(1) |
| C(26) | 19(1) | 19(1) | 20(1) | −3(1) | 4(1) | −5(1) |

TABLE S5
Hydrogen coordinates (×10⁴) and isotropic
displacement parameters (Å² × 10³)

| | x | y | z | U(eq) |
|---|---|---|---|---|
| H(8) | 5100 | 3931 | 6517 | 18 |
| H(9) | 4772 | 1086 | 6216 | 17 |
| H(13A) | 8908 | 1134 | 7326 | 39 |
| H(13B) | 8548 | −692 | 7065 | 39 |
| H(13C) | 8822 | 31 | 7219 | 51 |
| H(13D) | 8735 | 1929 | 7510 | 51 |
| H(15) | 6781 | −297 | 8613 | 27 |
| H(17A) | 4958 | 1043 | 8403 | 26 |
| H(17B) | 5315 | −825 | 8579 | 26 |
| H(18A) | 4682 | −1755 | 7405 | 24 |
| H(18B) | 4026 | −1091 | 7888 | 24 |
| H(19A) | 7125 | 2571 | 5650 | 37 |
| H(19B) | 6395 | 2304 | 4997 | 37 |
| H(19C) | 6810 | 698 | 428 | 37 |
| H(20A) | 4372 | 4694 | 4884 | 32 |
| H(20B) | 3530 | 4311 | 4369 | 32 |
| H(20C) | 3310 | 3304 | 4204 | 41 |
| H(20D) | 3968 | 4642 | 4567 | 41 |
| H(21A) | 4626 | 3178 | 3826 | 42 |
| H(21B) | 4054 | 1669 | 4020 | 42 |
| H(21C) | 4890 | 2039 | 4537 | 42 |
| H(21D) | 4421 | 1412 | 4429 | 42 |
| H(21E) | 4993 | 2921 | 4235 | 42 |
| H(21F) | 4157 | 2551 | 3718 | 42 |
| H(22A) | 1061 | 3682 | 4548 | 21 |
| H(22B) | 1317 | 3174 | 5391 | 21 |
| H(23A) | 1988 | 1642 | 4223 | 66 |
| H(23B) | 1306 | 771 | 4620 | 66 |
| H(23C) | 2189 | 1093 | 5063 | 66 |
| H(22C) | 2065 | 1440 | 5324 | 112 |
| H(22D) | 1234 | 2489 | 4004 | 112 |
| H(23D) | 1148 | 2365 | 4339 | 54 |
| H(23E) | 1229 | 490 | 4079 | 54 |
| H(23F) | 1995 | 1390 | 5168 | 54 |
| H(24A) | 1786 | 7400 | 5849 | 26 |
| H(24B) | 1662 | 8592 | 5022 | 26 |
| H(24C) | 1689 | 6883 | 5681 | 34 |
| H(24D) | 1788 | 8224 | 4953 | 34 |
| H(25A) | 489 | 8679 | 5691 | 43 |
| H(25B) | 273 | 7859 | 5017 | 43 |
| H(25C) | 396 | 6666 | 5488 | 43 |
| H(25D) | 283 | 6790 | 4750 | 43 |
| H(25E) | 499 | 7611 | 5424 | 43 |
| H(25F) | 376 | 8803 | 6274 | 43 |
| H(26A) | 3915 | −1252 | 6121 | 29 |
| H(26B) | 3411 | 469 | 6714 | 29 |
| H(26C) | 3183 | −852 | 7378(10) | 39(3) |
| HN1 | 1937(11) | 6530(20) | 7888(11) | 39(3) |
| HN2 | 2775(11) | 6410(20) | | |

1. An isolated stereoisomer of tritoqualine having the structure D1 of FIG. 2.

2. An isolated stereoisomer of tritoqualine having the structure D2 of FIG. 3.

3. A method for treating diseases or disorders resulting from elevated histamine levels comprising administering an effective amount of the isomer of claim 1 or 2 to a subject so as to treat disorders with elevated histamine levels.

4. A method of reducing histamine levels by inhibiting histidine decarboxylase comprising administering an effective amount of the isomer of claim 1 to a subject thereby reducing histamine levels.

5. A method of reducing histamine levels by inhibiting histidine decarboxylase comprising administering an effective amount of the isomer of claim 2 to a subject thereby reducing histamine levels.

6. A method for treating an immune system disease or disorder comprising administering an effective amount of the isomer of claim 1 to a subject so as to treat the immune system disease or disorder.

7. A method for treating an immune system disease or disorder comprising administering an effective amount of the isomer of claim 2 to a subject so as to treat the immune system disease or disorder.

8. The method of claim 6 or 7, wherein the immune system disease or disorder is an inflammatory disease or disorder.

9. The method of anyone of claim 4 or 5, wherein the isomer is in a salt form or hydrate form.

10. The method of anyone of claim 4 or 5, wherein the isomer is administered with a pharmaceutically acceptable carrier.

11-15. (canceled)

16. A method for treating diseases or disorders resulting from elevated histamine levels comprising administering an effective amount of the isomer of claim 1 to a subject so as to treat disorders with elevated histamine levels, wherein the disorder is selected from a group consisting of allergic rhinitis, dermatitis, atopic dermatitis, urticaria, pruritus, eczema, allergic erythema and non allergic erythema, food allergy, asthma, inflammatory bowel disease such Irritable bowel disease, crohn disease, celiac disease, gastristis, GERD, oesophagitis and dyspepsia.

17. A method for treating diseases or disorders resulting from elevated histamine levels comprising administering an effective amount of the isomer of claim 2 to a subject so as to treat disorders with elevated histamine levels, wherein the disorder is selected from a group consisting of allergic rhinitis, dermatitis, atopic dermatitis, urticaria, pruritus, eczema, allergic erythema and non allergic erythema, food allergy, asthma, inflammatory bowel disease such Irritable bowel disease, crohn disease, celiac disease, gastristis, GERD, oesophagitis and dyspepsia.

18. The method of claim 6 or 7, wherein the immune system disease or disorder is selected from a group consisting of atopic dermatitis, allergic dermatitis, inflammatory skin disorder, conjunctivitis, allergeric rhinitis, asthma, and allergy.
